(19) United States
(12) Patent Application Publication
(10) Pub. No.: US 2013/0210755 A1
(43) Pub. Date: Aug. 15, 2013

(54) METHODS FOR INHIBITING INSECT INFESTATIONS

(71) Applicants: Amy Louise Marr, Greenfield, IN (US); Jeffery Alan Meyer, Greenfield, IN (US); Katherine Ann Meyer, Anderson, IN (US); Jane Granville Owens, Indianapolis, IN (US); Tandy Elizabeth Paarlberg, Greenfield, IN (US); Daniel Earl Snyder, Indianapolis, IN (US); Joseph Raymond Winkle, Carmel, IN (US)

(72) Inventors: Amy Louise Marr, Greenfield, IN (US); Jeffery Alan Meyer, Greenfield, IN (US); Katherine Ann Meyer, Anderson, IN (US); Jane Granville Owens, Indianapolis, IN (US); Tandy Elizabeth Paarlberg, Greenfield, IN (US); Daniel Earl Snyder, Indianapolis, IN (US); Joseph Raymond Winkle, Carmel, IN (US)

(73) Assignee: ELI LILLY AND COMPANY, Indianapolis, IN (US)

(21) Appl. No.: 13/879,082

(22) PCT Filed: Nov. 7, 2011
(86) PCT No.: PCT/US11/59571

§ 371 Date: Apr. 12, 2013

Related U.S. Application Data
(60) Provisional application No. 61/410,396, filed on Nov. 5, 2010.

(51) Int. Cl. A61K 31/7048; A01N 43/22; A61K 45/06

(52) U.S. Cl. CPC A61K 31/7048; A61K 45/06; A01N 43/22
USPC 514/28

(57) ABSTRACT

Provided are 3 methods and formulations using feed-through administration and dosing of spinosyns for controlling biting or nuisance insects on animals, for controlling equine feces-dependent insects, and for preventing internal infestation of bot larvae in equine animals.

Manure piles provide an optimum and often essential environment for larval maturation of certain fly species. Feed-through pest control products for animals are known and used as dietary additives, which are eliminated in the animal's feces. The stages of the target pest's life cycle reliant on the manure are interrupted due to the presence of the feed-through product in the feces. Feed-through administration is normally chronic/daily in order to make sure all of the animal's feces contain sufficient product to disrupt the pest's life cycle, but is dosed preferably at the lowest possible amount in order to avoid any safety or environmental issues.

Four US marketed feed-through products for horses include Simplifly™ and Equitrol II™, made by Farnam and both containing diflubenzuron, an insect growth regulator (IGR). Solitude™ by Pfizer contains cyromazine, another type of IGR. Finally Equi-Fly™ by Med Vet Pharmaceuticals Ltd. contains the organophosphate tetrachlorvinphos also known as Rabon, a nerve toxin with associated potential for side effects in the target species and handlers.

While the use of these and other agents have been beneficial, alternative or improved formulations and methods are needed. Desirable formulations and methods would not only provide alternative therapies, but would also overcome at least some limitations of current therapies. Such limitations include toxicity and safety, environmental residues, efficacy (potency and duration), and resistance issues. Also impacting the beneficial use of therapies are administration obstacles, which include mode and recurrence of administration.

Provided are methods and formulations using feed-through administration and dosing of spinosyns for controlling equine feces-dependent insects (particularly the immature stages of such insects and those which bite or are a nuisance to the animal), for controlling biting or nuisance insects on mammals, and for preventing internal infestation of bot larvae in an equine animal. The methods and formulations overcome at least some of the limitations in the use of current agents. The methods comprise orally administering an effective feed-through amount of a spinosyn for a sufficient time to the mammal and/or equine animal. Also included are feed-through pharmaceutical formulations for the methods which comprises an effective feed-through amount of a spinosyn and a physiologically acceptable carrier. An additional benefit of the methods and formulations of the invention includes the reduction of the population of fly/insect vectors transmitting various diseases and parasites, which include but are not limited to conjunctivitis, Onchocerca cervicalis, Setaria spp, Thelazia lacrymalis, Habronema muscae, equine encephalitis viruses, and West Nile Virus.

Spinosyns are naturally derived fermentation products. They are macrolides produced by cultivation of Saccharopolyspora spinosa. The fermentation produces many factors, including spinosyn A and spinosyn D (also called A83543A and A8354D). Spinosyn A and spinosyn D are the two spinosyns that are most active as insecticides. A product comprised mainly of these two spinosyns (65-95% spinosyn A and 5-35% of spinosyn B) is available commercially under the common name “spinosad”, and is a preferred spinosyn agent. The major spinosyn factor, spinosyn A, is known to have an excellent human and animal safety and toxicological profile.

Each spinosyn has a 12-membered macrocyclic ring that is part of an unusual tetracyclic ring system to which two different sugars are attached, the amino-sugar forosamine and the neutral sugar 2N,3N,4N-(tri-O-methyl)rhamnose. This unique structure sets the spinosyns apart from other macrocyclic compounds.

Spinosyn A was the first spinosyn isolated and identified from the fermentation broth of S. spinosa. Subsequent examination of the fermentation broth revealed that S. spinosa produced a number of spinosyns that have been called spinosyns A to J (A83543A to J). The primary components are spinosyns A and D. Additional spinosyns, lettered from K to W, have been identified from mutant strains of S. spinosa. The various spinosyns are characterized by differences in the substitution patterns on the amino group of the forosamine sugar, at selected sites on the tetracyclic ring system and on the 2N,3N,4N-(tri-O-methyl)rhamnose group.

Boeck et al. described spinosyns A-H and J (which they called A83543 factors A, B, C, D, E, F, G, H and J), and salts thereof, in U.S. Pat. No. 5,362,634 (issued Nov. 8, 1994); U.S. Pat. No. 5,496,932 (issued Mar. 5, 1996); and U.S. Pat. No. 5,571,901 (issued Nov. 5, 1996). Mynderse et al. described spinosyns L-N (which they called A83543 factors L, M and N), their N-demethyl derivatives, and salts thereof, in U.S. Pat. No. 5,202,242 (issued Apr. 13, 1993); and Turner et al. described spinosyns Q-T (which they called A83543 factors Q, R, S and T), their N-demethyl derivatives, and salts thereof, in U.S. Pat. No. 5,591,606 (issued Jan. 7, 1997) and U.S. Pat. No. 5,631,155 (issued May 29, 1997). Spinosyns K, O, P, U, V, W and Y are described, for example, by Carl V. DeAmicis, James E. Dripps, Chris J. Hatton and Laura I. Karr in American Chemical Society's Symposium Series: Phytochemicals for Pest Control, Chapter 11, “Physical and Biological Properties of Spinosyns: Novel Macrolide Pest-Control Agents from Fermentation”, pages 146-154 (1997).

Spinetoram is the common name for a mixture of 25-90%, preferably 50-90% (2R,3aR,5aR,5bS,9S,13S,14R,16aS,16bR)-2-(6-deoxy-3-O-ethyl-2,4-di-O-methy-1-.alpha.-L-mannopyranosyloxy)-13-[(2R,5S,6R)-5-(dimethylamino)tetrahydro-6-methylpyran-2-yloxy]-9-ethyl-2,3,3a,4,5,5a,5b,6,9,10,11,12,13,14,16a,16b-hexadecahydro-14-methyl-1H-as-indaceno[3,2-d]oxacyclododecine-7,15-dione (referred to as “dihydro-Et-J”), and 10-75%, preferably 10-50% (2R,3aR,5aS,5bS,9S,13S,14R,16aS,16bS)-2-(6-deoxy-3-O-ethyl-2,4-di-O-methy-1-.alpha.-L-mannopyranosyloxy)-13-[(2R,5S,6R)-5-(dimethylamino)tetrahydro-6-methylpyran-2-yloxy]-9-ethyl-2,3,3a,5a,5b,6,9,10,11,12,13,14,16a,16b-tet-radecahydro-4,14-dimethyl-1H-as-indaceno[3,2-o]oxacyclododecine-7,15-dione (referred to as “Et-L”). (Podhorez et al., U.S. 2008/0108800A1). Spinetoram is described as providing long-lasting control of a broad spectrum of insect pests in a variety of crops (Dow AgroSciences Spinetoram Technical Bulletin, November 2006). Spinetoram has been registered in New Zealand as an insecticide in the pome fruit market (“Dow AgroSciences Receives First Global Registration for Spinetoram Insecticide,” Dow AgroSciences Newsroom, Corporate News, Aug. 10, 2007).

The term “spinosyn” or “spinosyn component” as used herein refers to an individual spinosyn factor (spinosyn A, B, C, D, E, F, G, H, J, K, L, M, N, O, P, Q, R, S, T, U, V, W or Y), an N-demethyl derivative of an individual spinosyn factor, a physiologically acceptable salt thereof, or a combination thereof. The terms also include spinetoram or a physiologically acceptable salt thereof.

The spinosyns can react to form salts that are also useful in the methods and formulations of this invention. The salts are prepared using standard procedures for salt preparation. For example, spinosyn A can be neutralized with an appropriate acid to form an acid addition salt. The acid addition salts of spinosyns are particularly useful. Representative suitable acid addition salts include salts formed by reaction with either an organic or inorganic acid such as, for example, sulfuric, hydrochloric, phosphoric, acetic, succinic, citric, lactic, maleic, fumaric, cholic, pamoic, mucic, glutamic, camphoric, glutaric, glycolic, phthalic, tartaric, formic, lauric, stearic, salicylic, methanesulfonic, benzenesulfonic, sorbic, picric, benzoic, cinnamic and like acids.

The term mammal has its normal meaning, and includes humans, dogs, cats, cattle, equine animals including horses, goats, sheep. Preferred target mammals are those which are in contact daily with biting and nuisance insects, such as horses, cattle, sheep, and goats.

An equine animal is a member of the family Equidae and includes horses, donkeys, and mules.

Equine feces-dependent insects are those insects which can or do depend upon, or reside in, equine manure for at least a portion of their life cycle, which includes use of the feces as a food or development source. Examples include, but are not limited to, bot flies, stable flies, and house flies. The equine feces are used by bot fly larvae as a means for transport out of the equine animal's body for further development. House and stable flies, for example, can lay their eggs in the feces.

Biting and/or nuisance insects include those pests which feed off the blood or external secretions of mammals. The secretions of mammals include eye lacrimations and wound exudates, for example. Examples of such insects includes mosquitoes, midges, horn flies, stable flies, deer flies, horse flies, and face flies.

There are nine different species of Gasterophilus globally, with three species which commonly affect equine animals in North America: Gasterophilus intestinalis, Gasterophilus nasalis, and Gasterophilus haemorrhoidalis. These three are often referred to as horse bots, and the larvae of all three infest the animal in the gastrointestinal tract. The duration of internal infestation is normally from two to twelve months. At the end of this internal infestation period, the larvae are expelled in the animal's feces, and remain there for a period of time prior to crawling out of the feces and finding a suitable location in the soil. The mature larva then molts into a pupa, and after a few weeks, the adult fly emerges from the soil.

Controlling, when used in the context of equine feces-dependent insects, refers to either ameliorating or eliminating insects from coming into contact with equine feces, killing insects if they ingest or spend sufficient time in, around, or on the equine feces, or killing or retarding growth of any present or developing insect (larva/maggots) in or on the equine feces.

Controlling, when used in the context of biting or nuisance insects on a mammal, refers to ameliorating or eliminating insects from coming into contact with the mammal, as well as killing the insects once they have ingested mammalian secretions.

Preventing, in the context of internal infestations of bot larvae in an equine animal, refers to ameliorating, reducing the level or amount of, or stopping an infestation in an equine animal host by hindering the ability of the bot larvae to effectively attach to the animal's gastrointestinal mucosa.

Effective amount, in the context of a spinosyn orally administered under a feed-through approach, refers to the amount sufficient to pass through into the animal's feces to provide a controlling effect on equine feces-dependent insects. Effective amount, in the context of the amount of spinosyn present in an equine animal's feces, is that amount sufficient to provide a controlling effect on equine feces-dependent insects. Effective amount in the context of controlling biting or nuisance insects with feed-through dosing and oral administration is that amount of spinosyn which will result in a sufficient amount of the spinosyn to be present in the mammal's secretions to control the biting or nuisance insects. Lastly, effective amount, in the context of preventing internal infestation of bot larvae in an equine animal using fed-through dosing and oral administration, is that amount sufficient to prevent internal bot larvae infestation. In all of the above, such amounts should result in no or few adverse events in the treated animal. As those familiar with the art will understand, these amounts will vary depending upon a number of factors. These factors include, for example, the type of equine animal or mammal being treated, its weight and general physical condition, and the dosing regimen. Ranges for spinosyns to be orally administered in a feed-through dosing regimen range from about 0.1 to about 10, desirably 0.2 to 5, and more desirably from about 0.4 to about 1, mg/kg of weight of the equine animal. Typically, the controlling/preventing effect will be obtained by chronic or daily administration, and on an on-going basis. Such an approach would be chronic administration, every day for at least one week, at least two weeks, at least a month, or twelve weeks or longer, in the ranges above on a daily basis. The amount of the spinosyn present in the animal's feces can be from 2 to 50 ppm, and more preferably 5 to 40 ppm of the spinosyn in the feces. The dose can be chronically administered at a level which is sub-optimal or completely or mostly non-efficacious levels for other purposes, such as internal pest control, but which will still provide the controlling effect on equine feces-dependent insects, biting and/or nuisance insects, and prevent bot larvae infestation. For example, for feed-through administration to prevent bot larvae, the amount administered could be less than about 10 mg/kg, and preferably less than about 1 mg/kg, of the weight of the equine animal.

Physiologically acceptable as used in this application, for example with reference to salts and formulation components such as carriers and ingredients, means relatively non-toxic and safe when administered to the equine animal or mammal.

The formulations and methods of this invention may further include, in combination with the spinosyn component, one or more other active ingredients that have activity against other pests. Examples of such include synthetic pyrethroids, natural pyrethins, organophosphates, organochlorines, carbamates, foramidines, avermectins, milbemycins, insect growth regulators (including chitin synthesis inhibitors, juvenile hormone analogs, and juvenile hormones), nitromethylenes, pyridines and pyrazoles.

Oral formulation means that the spinosyn component or components, either alone or in combination with one or more of the other types of compounds listed supra, is formulated into a product or formulation suitable for administering to the equine animal or mammal by mouth. These products or formulations include, but are not limited to, tablets, capsules, pellets, granules, mineral and protein supplement formulations, liquids, gels, pastes, oral sprays, buccal formulations, powders and animal feeds containing the active component or components. Generally, such formulations include a physiologically acceptable carrier. Such carriers are well known in the veterinary arts. The amount of the spinosyn in such an oral formulation may be from greater than 0% to 95%, desirably 0.1% to 60%, and more desirably 1% to 50%, all weight percentages. Carrier is used herein to describe any ingredient other than the active components in a formulation. The choice of carrier will to a large extent depend on factors such as the particular formulation, the effect of the carrier on solubility and stability, and the nature of the dosage form. Examples of carriers are well known in the art, and include excipients, diluents, stabilizers, and adjuvants.

The phrase feed-through oral dose formulation means an oral dose of spinosyn which when administered to the equine animal or mammal results in the desired effect, and provides the animal with the dose of the spinosyn as described above. The formulation is normally administered over a prolonged time, and/or for a time and at rate sufficient to result in an effective amount of spinosyn to be present in an equine animal's feces, a mammal's secretions, or internally in an equine animal. The phrase prolonged time or chronically comprises a period of administration normally at least the length of the relevant insect or fly season, with administration beginning a few weeks prior to the insects/flies becoming active, through the end of adult insect activity. The administration can be at least daily for 7 days, daily for a period of at least two weeks, preferably daily for at least 30 days, and more preferably for at least twelve weeks.

This invention relates to feed-through dose oral formulation, and its use in the methods described above through a feed-through approach, said formulation comprising an effective amount of a spinosyn, and a physiologically acceptable carrier, in an oral dosage form for feed-through administration. Also encompassed by the invention is the use of a spinosyn for the manufacture of a oral formulation for use in the methods described above, through a feed-through approach. The amount of spinosyn present in the feed-through dose oral formulation will be 0.1 to about 10 mg/kg, and more preferably be 0.4 to 5 mg/kg of the weight of the animal.

A study was conducted to evaluate the efficiency of oral spinosad feed-through for controlling development of immature stages of the house fly (Musca domestica) and stable fly (Stomoxys calcitrans) in the manure of treated horses. There were five treatment groups, having three horses each. Treatment groups 1-4 were administered the following pellet formulation:

Test Substance Formula

| Ingredient | Amount (lbs) | % by Weight | % Spinosad |
|---|---|---|---|
| Alfalfis Meal (17%) | 892.5 | 44.63 | — |
| Wheas Midds | 810.5 | 40.52 | — |
| *Spinosad 20% Premix | 175.0 | 8.75 | 1.5 |
| PM 42 (Kare Lass) | 80.6 | 4.00 | — |
| Mineral Oil | 40.0 | 2.00 | — |
| Myco Curb Dry (50) | 2.0 | 0.10 | — |
| Total | 2,000.0 | 100.00 | 1.5 |

Treatment groups 1-4 were provided the test substance top-dressed onto feed supplement and fed at a rate to deliver a dose, respectively, of approximately 0.6, 0.8, 1.0, and 1.2 mg spinosad/kg body weight/day, daily for the duration of the study. Treatment group 5 was the negative control group and was fed the above formulation without spinosad, and each horse in this treatment group received one ounce (28.3 g) of the negative control substance.

Fecal samples from the three horses in each treatment group were collected on study days—1, 3, 10, and 17, and bioassayed with eggs of house flies and stable flies, and the number of adult flies that emerged from the fecal sample bioassays were determined. The concentration of spinosyn A in the fecal samples was also determined.

Results from the study showed administration of spinosad per above was safe and highly efficacious in preventing emergence of adult house flies and stable flies from horse manure in all treated groups, and resulted in no observed adverse effects. Particularly, all 4 spinosad treatment groups were 100% effective in preventing emergence of adult house flies in all three post-administration collection days. Reduction in emergence of stable fly adults was 100% for all 4 spinosad treatments at all three post-administration collections, with one exception. At the study day 17 fecal sample collection, three stable fly adults emerged (out of 30 eggs added to the three replicates of the sample for a total of 90 eggs) from the fecal sample collected from one horse in the 0.6 mg spinosad/kg of body weight group, resulting in 97.25% reduction in stable fly emergence for this treatment group on this fecal sample collection day. The concentration of spinosyn A quantified in the fecal samples for spinosad treated horses ranged from a low of 0.9 ppm in a sample from one horse in the 0.6 mg spinosad/kg of body weight group on study day 3, to a high of 8.6 ppm in a sample from one horse in the 1.2 mg spinosad/kg of body weight group on study day 10.

The mean spinosyn A concentration in fecal samples collected from all three animals in treatments groups 1-3 was similar, ranging from a low of 2.9 ppm for the 0.8 mg spinosad/kg of body weight group to a high of 3.4 ppm for the 0.6 mg spinosad/kg of body weight group. The mean concentration of spinosyn A in fecal samples from the 1.2 mg spinosad/kg of body weight group was greater than the other three treatment groups, and was 5.5 ppm.

Another study was carried out, similar to the above study, with a few changes. There were three spinosad administered groups, with the rates of spinosad being 0.4, 0.5, and 0.6 mg spinosad/kg of body weight. There was a positive control group, administered between 0.6 and 0.7 mg cyromazine/kg of body weight. There was also a negative control group.

There were no observed adverse effects in any of the treatment groups. All three spinosad administered groups and the cyromazine administered group had 100% efficacy in preventing emergence of adult house flies at all three fecal sample collection dates. Reduction of the emergence of stable flies was 100% for all three spinosad administered groups and the cyromazine administered group, with one exception. At the study day 10 fecal sample collection, one stable fly adult emerged (out of 30 eggs add to the three replicates of the sample for a total of 90 eggs) from the fecal sample collected from one horse in the 0.4 mg spinosad/kg of body weight group, resulting in 99.3% reduction in stable fly emergence for this treatment group on this fecal sample collection day. The mean spinosyn A concentration in fecal samples collected from all three animals at all three post-administration dates for the three spinosad treatment groups was 2.2, 3.2, and 3.7 ppm, respectively.

The following study in cattle demonstrates that spinosad feed-through can have a systemic effect in controlling biting or nuisance insects. These data suggest spinosad feed-through can attain sufficient systemic concentrations to kill biting flies following administration of a low oral dose. A study was conducted to evaluate the efficacy of orally administered spinosad to control horn flies (Haematobia irritans), an obligate ectoparasite of cattle. A liquid spinosad formulation was topically applied to a palatable feed ration and fed to cattle at doses of 0.03, 0.10, 0.30 and 3.0 mg/kg of body weight. The number of horn flies remaining after 24 hours exposure to the cattle in an enclosed room was determined on test days 2, 4, 9, 11, 16, 18, 23 and 25 and was compared to the number remaining for the control group. The percent reduction in horn flies was notable for the 0.30 and 3.0 mg/kg test groups. Horn flies were reduced by at least 89.2% for the 3.0 mg/kg test group at all time points. Horn flies were reduced by at least 63.3% for the 0.30 mg/kg test group from day 9 and on.

Thus it follows that spinosad feed through may control biting insects such as mosquitoes, midges, horn flies, stable flies, deer flies and horse flies feeding on treated animals. Many horses development hypersensitivity reactions to biting insects, particularly Culicoides, a type of midge (e.g., sweet itch). Spinosad may reduce the severity of this disorder through its systemic effect. In addition to controlling biting insects, spinosad feed through may control face flies and other nuisance flies which feed on eye lacrimations and exudates from wounds. More importantly, spinosad as a feed through may effectively reduce the population of insect vectors transmitting various infectious diseases and parasites, not limited to conjunctivitis, Onchocerca cervicalis, Setaria spp, Thelazia lacrymalis, Habronema muscae Equine Infectious Anemia, Equine Encephalitis Viruses, and West Nile Virus.

1-55. (canceled)

56. A method of controlling an equine feces-dependent insect which comprises orally administering an effective feed-through amount of a spinosyn to an equine animal for a sufficient time, wherein said administration is in the form of a tablet, capsule, bolus, solution, paste, gel, feed, premix, suspension, or elixir, and said effective feed-through amount results in said spinosyn being present in said feces of said equine animal in an amount of about 2 to about 50 ppm.

57. The method of claim 56, wherein said amount is between about 5 to about 40 ppm.

58. The method of claim 56, wherein said equine animal is a horse.

59. The method of claim 56, wherein said spinosyn is administered in an amount of between about 0.1 and about 10 mg/kg of equine animal body weight.

60. The method of claim 59, wherein said amount is from about 0.1 to about 1 mg/kg of equine animal body weight.

61. The method of claim 56, wherein said equine feces-dependent insect is a bot larva selected from Gasterophilus intestinalis, Gasterophilus nasalis, or Gasterophilus haemorrhoidalis.

62. The method of claim 56, wherein said administration is daily for at least 7 consecutive days.

63. The method of claim 62, wherein said administration is daily for at least twelve weeks.

64. The method of claim 56, wherein said spinosyn is administered with at least one other active ingredient.

65. The method of claim 56, wherein said spinosyn is spinosad or a physiologically acceptable salt thereof.

66. The method of claim 56, wherein said spinosyn is spinetoram or a physiologically acceptable salt thereof.

67. A feed-through dose oral formulation for a mammal comprising an effective feed-through amount of a spinosyn, and a physiologically acceptable carrier, in an oral dosage form adapted for feed-through administration, wherein said formulation comprises about 0.1 to about 10 mg of the spinosyn per kg of body weight of said mammal.

68. The formulation of claim 67, wherein said mammal is a horse.

69. The formulation of claim 67, wherein said amount is from about 0.1 to about 1 mg/kg of equine animal body weight

70. The formulation of claim 67, wherein said formulation comprises an additional active ingredient.

71. The formulation of claim 67, wherein said spinosyn is spinosad or a physiologically acceptable salt thereof.

72. The formulation of claim 67, wherein said spinosyn is spinetoram or a physiologically acceptable salt thereof.

73. A method of controlling biting or nuisance insects on, or preventing internal infestation of bot larvae in, a mammal comprising orally administering a feed-through amount of a spinosyn to said mammal for a sufficient time, wherein said feed-through amount is between about 0.1 and about 10 mg/kg of mammal body weight.

74. The method of claim 73, wherein said mammal is a horse.

75. The method of claim 73, wherein at least one other active ingredient is administered to said mammal.

76. The method of claim 73, wherein said administration is in the form of a tablet, capsule, bolus, solution, paste, gel feed, premix, suspension, or elixir

77. The method of claim 73, wherein said administration is daily for at least 7 consecutive days.

78. The method of claim 73, wherein said administration is carried out chronically.

79. The method of claim 73, wherein said biting or nuisance insect is selected from mosquitoes, midges, horn flies, stable flies, deer flies, horse flies, and face flies.

80. The method claim 73, wherein said spinosyn is spinosad or a physiologically acceptable salt thereof.

81. The method of claim 73, wherein said spinosyn is spinetoram or a physiologically acceptable salt thereof.
